(19) United States
(12) Patent Application Publication
(10) Pub. No.: US 2024/0408077 A1
(43) Pub. Date: Dec. 12, 2024

(54) ATM KINASE INHIBITORS FOR USE IN THE TREATMENT OF NEUROLOGICAL CONDITIONS

(71) Applicant: The University of Birmingham, Birmingham (GB)

(72) Inventors: Zubair Ahmed, Birmingham (GB); Richard Ian Tuxworth, Birmingham (GB)

(21) Appl. No.: 18/700,771

(22) PCT Filed: Oct. 13, 2022
(86) PCT No.: PCT/GB2022/052607

§ 371 Date: Apr. 12, 2024

(30) Foreign Application Priority Data
Oct. 14, 2021 (GB) 2114704.6

(51) Int. Cl. A61K 31/4745; A61P 25/00

(52) U.S. Cl. CPC A61K 31/4745; A61P 25/00

(57) ABSTRACT

The present disclosure relates to the use of ATM inhibitors, such as AZ1390 and related compounds, in the treatment of various neurological conditions, by protecting against or treating neuronal damage or neuronal degeneration, including traumatic injury, such as spinal cord injury (SCI).

### FIELD OF THE DISCLOSURE

The present disclosure relates to the use of ATM inhibitors, such as AZ1390 and related compounds, in the treatment of various neurological conditions, including traumatic injury, such as spinal cord injury (SCI).

### BACKGROUND OF THE DISCLOSURE

Spinal cord injury (SCI) is a debilitating condition that affects between 200,000 to 1.2 million people every year (1). SCI affects mainly young people, with the biggest cause being motor vehicle accidents. Most people survive the initial injury and but require life-long care which places a significant economic burden on society in general. At present, there are no cures for SCI. The only drug that is licensed for use in SCI is Lyrica (pregabalin) but this is only a palliative treatment for SCI-induced neuropathic pain and does not combat the underlying pathological changes after injury. The treatment of SCI is complicated further by the low intrinsic capacity of CNS neurons to re-grow after injury and the presence of extrinsic factors such as myelin- and scar-derived inhibitory molecules in the environment of the regenerating axon (2,3).

Double strand breaks (DSBs) in DNA are genotoxic and can lead to genome instability in replicating cells and, if unrepaired, can trigger apoptosis (4). In post-mitotic neurons, which are non-replicating and cannot easily be replaced, unrepaired DSBs are potentially more damaging. DSBs are sensed by the Ku70/80 or the MRN complex, composed of Mre11, Rad50 and NBS1/Nbn, with repair via non-homologous end-joining (NHEJ) or homologous recombination (HR). The ataxia telangiectasia mutated (ATM) and related ataxia telangiectasia and Rad3-related (ATR) kinases mediate many of the cellular events occurring as a consequence of DSB, such as cell-cycle arrest, repair and apoptosis (5, 6). ATM is particularly important for efficient repair of DSBs in heterochromatin by the NHEJ machinery. In contrast, ATR is required to overcome replication stress via HR (5, 6). HR is not likely to be available to post-mitotic neurons as it requires a sister chromatid as template.

We have shown recently that the ATM pathway is activated in dorsal root ganglion neurons (DRGN) after SCI and that suppression of ATM or a key downstream target, Checkpoint kinase-2 (Chk2) using small molecule pharmacological inhibitors promotes recovery of function after SCI (7). KU-60019, an ATM inhibitor reduced γH2Ax⁺ foci (a common way to monitor direct activation of the DNA damage pathway (8)) and promoted significant functional recovery after SCI, but required twice weekly injections, directly into the cerebrospinal fluid (CSF) (7).

Recently, the pharmacodynamic/pharmacokinetic profile of a CNS-penetrant, highly potent (cell IC₅₀=0.78 nM) and highly selective (>10,000-fold over kinases within the same family), orally bioavailable ATM inhibitor, AZD1390, was described (9). Significant concentrations of AZD1390 were present in the brain after oral administration and bioavailable for at least 8 hours (9). In syngeneic and patient-derived brain gliomas established in mice, AZD1390 dosed in combination with daily ionizing radiation therapy induced tumor regression and prolonged survival, suggesting clinical development of this compound for use as a radiosensitizer in CNS malignancies (9).

### SUMMARY OF THE DISCLOSURE

The present disclosure is based on studies using a class of brain penetrant ATM inhibitors that inhibit ATM function and as a result promote neuronal survival, axon regeneration and promote recovery of function. As the inhibitors are brain penetrant, they are able to access the CNS, without having to administer directly to the CNS, in suitable concentrations to promote significant functional recovery after, for example, SCI. However, SCI shares many of the same pathological features as other neurological diseases such as traumatic brain injury (TBI), stroke and eye injury and is therefore relevant to all of these, as well as other neurological conditions.

In a first teaching, there is provided a pharmaceutical formulation for use in a method of treating a neurological condition, by protecting against or treating neuronal damage or neuronal degeneration, the pharmaceutical formulation comprising a compound of Formula (I):

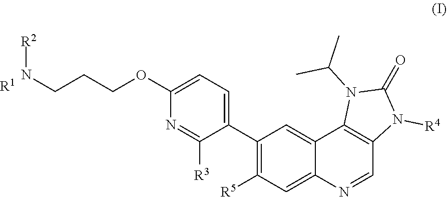

or a pharmaceutically acceptable salt, solvate, ester or other physiologically acceptable functional derivative thereof, where:
R¹ is methyl;
R² is hydro or methyl; or R¹ and R² together with the nitrogen atom to which they are bonded
form an azetidinyl, pyrrolidinyl or piperidinyl ring;
R³ is hydro or fluoro;
R⁴ is hydro or methyl; and
R⁵ is hydro or fluoro; and
at least one pharmaceutically acceptable excipient,
wherein the pharmaceutical formulation is not applied directly to the central nervous system (CNS) and/or peripheral nervous system (PNS).

In one embodiment, R¹ and R² together with the nitrogen atom to which they are bonded form a piperidinyl ring.

In one embodiment, R³ is hydro.

In one embodiment, R⁵ is fluoro.

In one embodiment, R⁴ is methyl.

In one embodiment the compound is 8-[2-Fluoro-6-[3-(I-piperidyl)propoxy]-3-pyridyl]-1-isopropyl-3-methyl-imidazo[4,5-c]quinolin-2-one (AZD1390).

In a further aspect, there is provided a pharmaceutical formulation as described herein for use in a method of promoting neuronal regeneration. The neuronal regeneration may for example, be used to treat any neurological conditions disclosed herein. The pharmaceutical formulation may, for instance be used to promote neuronal regeneration after injury. The pharmaceutical formulation may function to inhibit or suppress ATM activation.

The neuronal damage or degeneration is typically damage or degeneration that occurs in any one or more of the neurological disorders mentioned herein.

The neurological condition may affect the CNS and/or PNS. The neurological condition may affect, for example, the spinal cord, brain and/or optic nerve. The neurological condition may be sporadic and/or inherited.

The neurological condition may result from neuronal damage. The neuronal damage may be caused, for example, by physical means and/or by chemical means. The physical means may result from, for example, surgery or trauma. Types of trauma may include, for example, blunt force, penetration, compression, pressure, and/or blast trauma. The surgery may be resection, and types of brain/spinal cord/eye surgery and other surgeries that may result in damage to the CNS or PNS. The chemical means may be a drug, neurotoxin, infection, inflammation, oxidative stress, or nitrosative stress, for example.

Protecting against, treating neuronal damage or neuronal degeneration and/or promoting neuronal regeneration may include one or more of, protection of neural cells from apoptosis, promoting survival of neural cells, increasing the number of neural cell neurites, increasing neurite outgrowth, promoting retinal gliosis, promoting regeneration of neural cells and increasing or stimulation of neurotrophic factors in the nervous system.

The disclosure concerns, in some embodiments, preventing and/or treating a neurological condition, such as spinal cord injury, optic nerve trauma and other related neurological condition, where the formulations are able to be administered such that the active agent is delivered via the blood, to the brain or nervous system and is capable of traversing the blood brain barrier (BBB). One issue with treating neurological conditions is that if a compound, which is to be used to treat the neurological condition, is unable to cross the BBB, the compound needs to be administered by a more direct route, such as by intrathecal or direct injection to the brain. Such administration is generally complex and has to be carefully managed. Thus, being able to administer a formulation to treat a neurological condition, which does not require the active agent to be administered by a manner that avoids delivery of the active agent via the BBB, would be advantageous. Thus, not applying the pharmaceutical formulations of the present disclosure directly to the CNS and/or PNS is understood to mean that the formulations, as described herein, are administered to a subject in a manner such that the active agent of the pharmaceutical gets to its site of action (e.g. brain, spinal cord, optic nerve, etc, via the blood, rather than being directly administered to the site of action. Thus, in some embodiments of the present disclosure, the pharmaceutical formulations may be administered orally, parenterally (e.g. intravenously, intra-arterially, intramuscularly, or subcutaneously), topically, nasally, pulmonarily, sublingually, vaginally, or rectally, for example. In one embodiment, the pharmaceutical formulation is administered orally.

The CNS in accordance with the present invention is understood to mean the brain, spinal cord and cerebral spinal fluid, retina, optic nerve and olfactory nerves. The PNS is understood to mean the nerves and ganglia outside the brain and spinal cord.

In a further teaching, there is provided a method of protecting against, preventing, or reducing development of a neurological condition, or treating, such as by promoting neuronal regeneration, a subject suffering from a neurological condition, such as spinal cord injury, optic nerve trauma and other related neurological conditions, the method comprising administering a pharmaceutical formulation as described herein to the subject in an amount sufficient to ameliorate or alleviate the condition.

Treatment may or may not be curative in the sense of returning a subject to a state prior to suffering from the condition. Thus, treatment may slow or halt disease progression for example, or may protect a subject from developing a condition, for example.

In one embodiment, the disclosure may relate to the treatment of traumatic neurological injuries, such as TBI and chronic traumatic encephalopathy (CTE); traumatic injury to the spinal cord or eye caused by e.g. blunt force, puncture, compression, concussive damage or ballistic damage; Ischaemia affecting the central nervous system (for the avoidance of doubt this does not refer to ischemic damage to the heart); and traumatic injury to the peripheral nervous system, such as traumatic injury affecting motor, sensory or autonomic nerves, and traumatic injury affecting peripheral glia.

Where the neuronal damage is due to trauma, this includes physical trauma as caused by a subject receiving physical damage to the neural tissue due to an external force, or material penetrating the neural tissue, as well as physical trauma to the head in general, which can further lead to associated problems in the spinal cord, brain (such as traumatic brain injury and chronic traumatic encephalopathy) or eye. Neuronal damage may also be indirect as in the case of chemotherapy, which results for example, in chemotherapy-induced neuropathic pain (CIPN). Additional traumatic conditions associated with the eye include retinal ischemia, acute retinopathy associated with trauma, postoperative complications, traumatic optic neuropathy (TON); and damage related to laser therapy (including photodynamic therapy (PDT)), damage related to surgical light-induced iatrogenic retinopathy, and damage related to corneal transplantation and stem cell transplantation of ocular cells.

Traumatic optic neuropathy (TON) refers to acute damage of the optic nerve secondary to trauma of the eye in general. Optic nerve axons can be directly or indirectly damaged, and vision loss can be partial or complete. Indirect damage to the optic nerve is typically caused by a force transfer from blunt head trauma to the nerve cervical canal. This is in contrast to direct TON resulting from anatomical destruction of optic nerve fibers from penetrating orbital trauma, bone fragments within the neural transluminal tube, or schwannoma. Patients who have received corneal transplants or ocular stem cell transplants can also suffer trauma.

In one embodiment, the disclosure may relate to the treatment of recessive neurodegenerative disorders and PNS disorders, such as amyotrophic lateral sclerosis-frontal temporal dementia spectrum disorders (ALS-FTD), including forms associated with an expansion of the C9orf72 locus and spinal muscular atrophy; lysosomal storage disorders with a neurological association such as neuronal ceroid lipofuscinosis (NCL); and neurodegenerative disorders affecting the peripheral nervous system, such as Charcot-Marie-tooth disease, acute motor axonal neuropathy, diabetic neuropathy and Guillain-Barre syndrome.

The disclosure may also relate to prevent neuronal dysfunction and to maintain neuronal function, for example in the treatment of Chemotherapy-induced neuropathic pain and Chronic ophthalmic disorders, such as glaucoma, age-related macular degeneration and diabetic retinopathy.

The formulations described herein may also be administered in advance of, or during, surgery, in order to protect the neural tissue, such as to protect the spinal cord or optic nerve from damage, which may occur as a result of surgery. Thus, the present disclosure also extends to prophylactic uses of the pharmaceutical formulations described herein, in a subject, particularly in advance or concurrently with decompressive/resection/reparative surgery, for example surgery which is conducted on the spine or eye, to correct acute or chronic damage, or surgery conducted on the brain, for example removal of tumours.

The present formulations and claimed compounds work by inhibiting ATM kinase. In order to be beneficial to a subject, it may not be necessary to completely inhibit ATM kinase activity in a subject. Thus, in some embodiments, the present disclosure is directed to partial inhibition of ATM kinase activity. By partial inhibition is meant that at least 30%, 40%, 50%, 60%, 70% 80% or 90% inhibition of ATM kinase may be sufficient. This may facilitate a skilled addressee in determining how much of a compound of formula (I) needs to be administered to a subject in need. It is possible to test a level of activity of ATM kinase in a sample from a subject and to add an amount of a compound of formula (I) to the sample, in order to determine a level of reduction in ATM kinase activity, following compound addition. The skilled addressee is then able to estimate how much of the compound should be administered to the subject based on this. ATM kinase activity may be assessed in accordance with the assays described in Pike et al., 2018 (10).

Alternatively, an amount of compound may be administered to a subject and their ATM kinase activity level determined after a period of time (such as 1 week, 2 weeks, 1 month or longer after administering the compound to the subject). Depending on the level of ATM kinase determined, the skilled addressee is able to ascertain whether or not the amount if compound which has been administered to the subject, is sufficient to reduce ATM kinase activity by a desired amount, as described above, and hence whether the amount of compound being administered to the subject is appropriate to achieve the desired level of ATM kinase inhibition, or if the dose needs to be increased, or can in fact be decreased.

### DETAILED DESCRIPTION

The present invention will now be further described with reference to examples and the figures, which show:

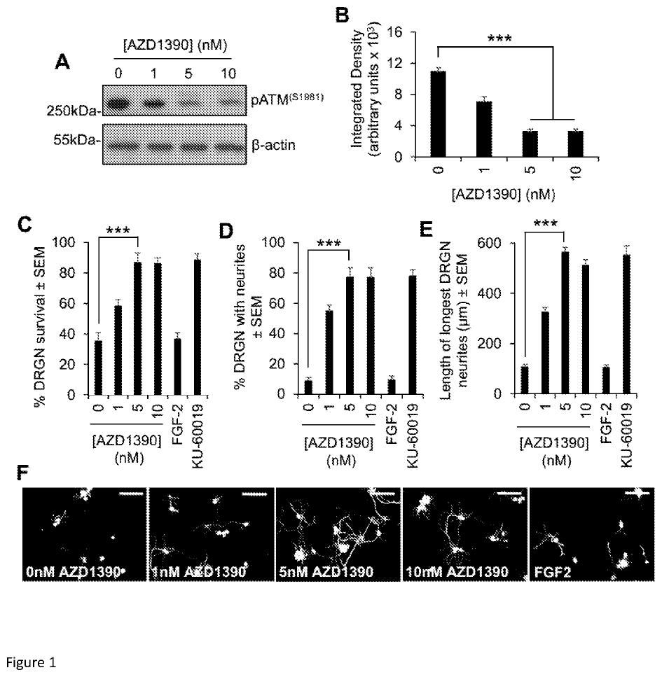

FIG. 1. AZD1390 suppresses pATM levels and promotes significant dorsal root ganglion neuron (DRGN) neurite outgrowth in the presence of inhibitory CNS myelin. (A) Western blot and (B) densitometry to show that increasing concentrations of AZD1390 suppresses pATM levels. Suppression of pATM promotes (C) DRGN survival, (D) % DRGN with neurites and (E) the mean longest DRGN neurite length despite the presence of inhibitory concentrations of CNS myelin extracts. (F) Representative photomicrographs to show neurite outgrowth from each condition. FGF2 was used as a positive control. KU-60019 also promoted similar levels of DRGN survival and neurite outgrowth as AZD1390. Scale bars in (F)=50 μm. n=3 wells/condition, 3 independent repeats (total n=9 wells/condition). ***=P<0.0001, one-way ANOVA with Dunnett's post hoc test.

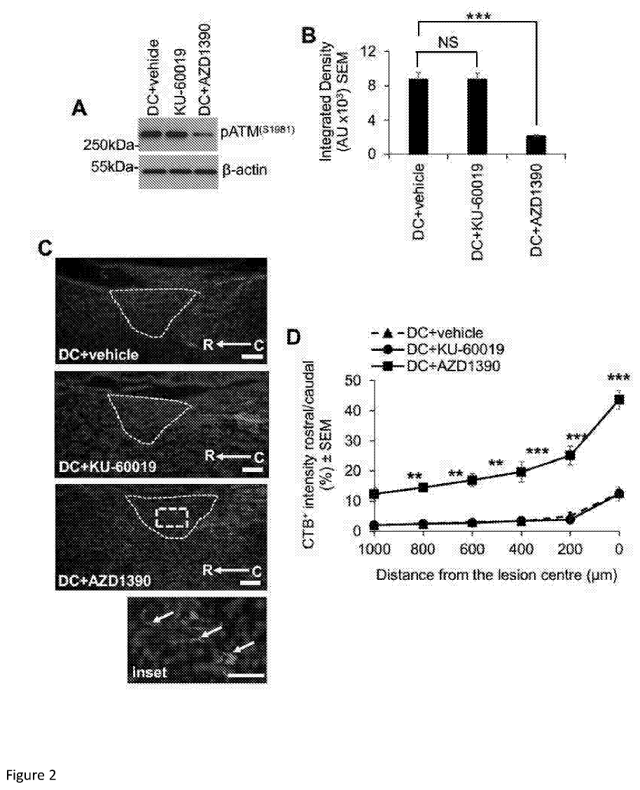

FIG. 2. Suppression of pATM with oral AZD1390 promotes dorsal column (DC) axon regeneration. (A) Western blot and (B) densitometry to show that oral AZD1390 significantly suppresses pATM levels in DRG at 4 weeks after DC injury. KU-60019 on the other hand, did not alter pATM levels by oral delivery. (C) CTB⁺ axons (red; nuclei=blue (stained with DAPI)) and (D) quantification to show that only oral AZD1390 treatment promoted axon regeneration through the lesion site and entry into the rostral cord. Fewer axons were present in the caudal cord in DC+vehicle and DC+oral KU-60019-treated animals and axons terminated at the lesion edge of the caudal cord. Inset in (C) shows high power magnification of the boxed region indicating CTB⁺ axons (arrows) growing through the lesion site towards the rostral cord. Dotted while lines show injury site. C to R=caudal to rostral. Scale bars in (C)=200 μm. n=6 mice/condition, 2 independent repeats (total n=12 mice/group). **=P<0.001; ***=P<0.0001, one-way ANOVA with Dunnett's post hoc test.

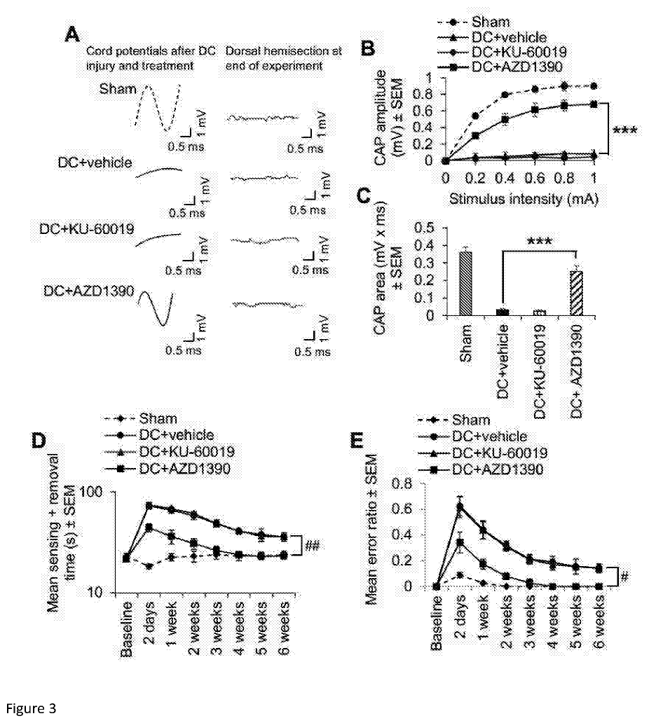

FIG. 3. Suppression of pATM with AZD1390 promotes electrophysiological, sensory and locomotor function recovery after DC injury. (A) Representative Spike 2 processed compound action potential (CAP) traces after oral delivery of ATM inhibitors. The normal CAP trace is ablated after DC injury and significantly restored by oral AZD1390 but not by oral KU-60019. Oral AZD1390 significantly improved (B) CAP amplitudes and (C) CAP areas at 6 weeks after DC injury. Only oral AZD1390 significantly improved (D) tape sensing and removal times and (E) ladder crossing performance (locomotor function) over 6 weeks after DC injury. n=6 mice/group, 2 independent experiments (total n=12 mice/group). ***P=0.0001, one-way ANOVA with Dunnett's post hoc test. #=P=0.00011, linear mixed models; ##=P=0.00012, generalized linear mixed models.

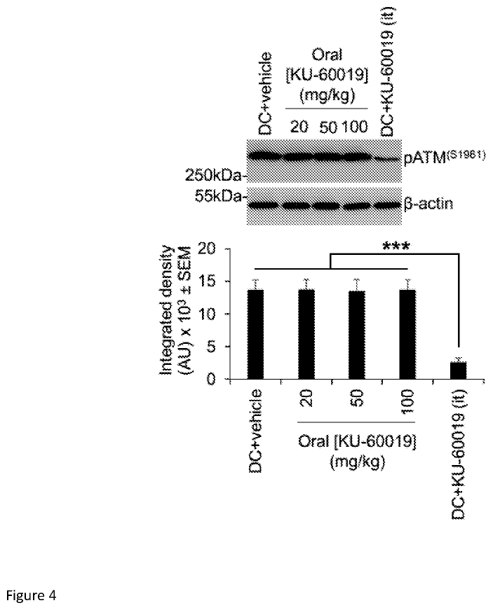

FIG. 4. Intrathecal but not oral KU-60019 suppresses pATM. (A) Western blot and (B) densitometry to show that oral KU6000-19 had no effect on pATM levels but intrathecal delivery of a previously published concentration of KU-60019, promoted 81% inhibition of pATM.

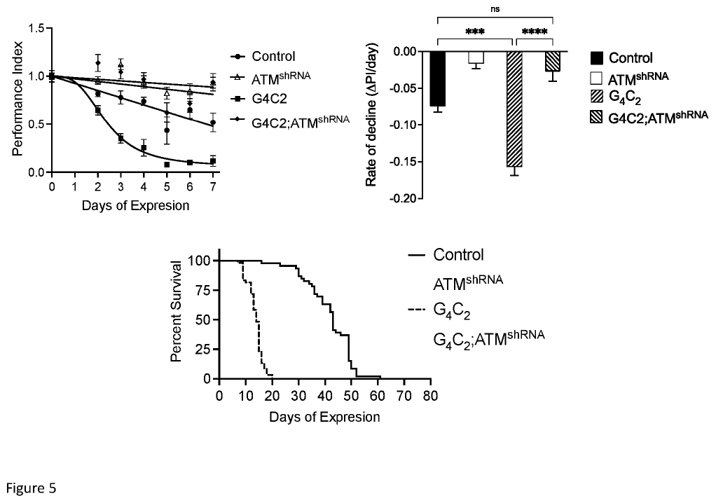

FIG. 5. Targeting ATM expression is protective in an in vivo model of amyotrophic lateral sclerosis-frontotemporal dementia (ALS-FTD) A. A longitudinal assay of the startle response of flies to a vibration stimulus quantified by automated tracking. G₄C₂-expressing flies show rapid decline in movement. Knockdown of ATM expression in adult neurons by shRNA prevents the decline.

B. Quantification of the rate of decline in the startle response in (A). Linear regression lines were plotted for each genotype and the slopes compared by ANOVA. *** p<0.001; **** p<0.0001; ns: p>0.05
C. Survival analysis. Expression of G₄C₂ causes to flies die early. Survival is rescued by knockdown of ATM expression in adult neurons by shRNA (G₄C₂ vs. G₄C₂;ATM^{shRNA} median survival 14 vs. 46 days, p<0.0001, Log Rank).

The pharmaceutical formulations as described herein may include a compound of formula (I), as the only active agent, which is administered to the subject, or may be administered in combination with one or more further active agents. An “active agent” means a compound (including a compound disclosed herein), element, or mixture that when administered to a patient, alone or in combination with another compound, element, or mixture, confers, directly or indirectly, a physiological effect on the subject. The indirect physiological effect may occur via a metabolite or other indirect mechanism.

An amount of compound according to formula (I) or any other active agents, would be at the discretion of the physician who would select dosages using his common general knowledge and dosing regimens known to a skilled practitioner.

Where a compound of formula (I) is administered in combination therapy with one, two, three, four or more, preferably one or two, preferably one other therapeutic agent, the compounds can be administered simultaneously or sequentially. When administered sequentially, they can be administered at closely spaced intervals (for example over a period of 5-10 minutes) or at longer intervals (for example 1, 2, 3, 4 or more hours apart, or even longer period apart where required), the precise dosage regimen being commensurate with the properties of the therapeutic agent(s).

Pharmaceutical formulations comprising the compounds of formula (I) may also be administered in conjunction with non-active agent treatments such as, photodynamic therapy, gene therapy; surgery.

The subject is typically an animal, e.g. a mammal, especially a human.

By a therapeutically or prophylactically effective amount is meant one capable of achieving the desired response, and will be adjudged, typically, by a medical practitioner. The amount required will depend upon one or more of at least the active compound(s) concerned, the patient, the condition it is desired to treat or prevent and the formulation of order of from 1 μg to 1 g of compound per kg of body weight of the patient being treated.

Different dosing regimens may likewise be administered, again typically at the discretion of the medical practitioner. Pharmaceutical formulations of the disclosure, may be provided by daily administration although regimes where the compound(s) is (or are) administered more infrequently, e.g. every other day, weekly or fortnightly, for example, are also embraced by the present disclosure.

By treatment is meant herein at least an amelioration of a condition suffered by a patient; the treatment need not be curative (i.e. resulting in obviation of the condition). Analogously references herein to prevention or prophylaxis herein do not indicate or require complete prevention of a condition; its manifestation may instead be reduced or delayed via prophylaxis or prevention according to the present disclosure.

The formulations for use in methods according to the present disclosure, may include the compound itself or a physiologically acceptable salt, solvate, ester or other physiologically acceptable functional derivative thereof. These are presented as a pharmaceutical formulation, comprising the compound or physiologically acceptable salt, ester or other physiologically functional derivative thereof, together with one or more pharmaceutically acceptable carriers therefor and optionally other therapeutic and/or prophylactic ingredients. Any carrier(s) are acceptable in the sense of being compatible with the other ingredients of the formulation and not deleterious to the recipient thereof.

Examples of physiologically acceptable salts of the compounds according to formula (I) include acid addition salts formed with organic carboxylic acids such as acetic, lactic, tartaric, maleic, citric, pyruvic, oxalic, fumaric, oxaloacetic, isethionic, lactobionic and succinic acids; organic sulfonic acids such as methanesulfonic, ethanesulfonic, benzenesulfonic and p-toluenesulfonic acids and inorganic acids such as hydrochloric, sulfuric, phosphoric and sulfamic acids.

Physiologically functional derivatives of compounds of formula (I) are derivatives, which can be converted in the body into the parent compound. Such physiologically functional derivatives may also be referred to as “pro-drugs” or “bioprecursors”. Physiologically functional derivatives of compounds of the present disclosure include hydrolysable esters or amides, particularly esters, in vivo. Determination of suitable physiologically acceptable esters and amides is well within the skills of those skilled in the art.

It may be convenient or desirable to prepare, purify, and/or handle a corresponding solvate of the compounds of formula (I) described herein, which may be used in the any one of the uses/methods described. The term solvate is used herein to refer to a complex of solute, such as a compound or salt of the compound, and a solvent. If the solvent is water, the solvate may be termed a hydrate, for example a mono-hydrate, di-hydrate, tri-hydrate etc, depending on the number of water molecules present per molecule of substrate.

It will be appreciated that the compounds of formula (I) may exist in various stereoisomeric forms and the compounds of formula (I) as hereinbefore defined include all stereoisomeric forms and mixtures thereof, including enantiomers and racemic mixtures. The present disclosure includes within its scope the use of any such stereoisomeric form or mixture of stereoisomers, including the individual enantiomers of the compounds of formula (I) as well as wholly or partially racemic mixtures of such enantiomers.

The compounds of the present disclosure may be purchased from commercial suppliers, or prepared using reagents and techniques readily available in the art. Suitable methods for making the compounds of formula (I) are described in WO2017046216, to which the skilled reader is directed and the entire contents of which are hereby incorporated by way of reference.

Pharmaceutical formulations include those suitable for oral, topical (including dermal, buccal and sublingual), rectal or parenteral (including subcutaneous, intradermal, intramuscular and intravenous), nasal and pulmonary administration e.g., by inhalation. The formulation may, where appropriate, be conveniently presented in discrete dosage units and may be prepared by any of the methods well known in the art of pharmacy. Methods typically include the step of bringing into association an active compound with liquid carriers or finely divided solid carriers or both and then, if necessary, shaping the product into the desired formulation. The present disclosure is based on the ability of the compounds of formula (I) being capable of crossing the BBB and being able to be administered by methods which allow the compound to be delivered to the site of action by crossing the BBB.

The oral formulations according to the present disclosure may, where appropriate, be conveniently presented in discrete dosage units and may be prepared by any of the methods well known in the art of pharmacy. Methods typically include the step of bringing into association an active compound with liquid carriers or finely divided solid carriers or both and then, if necessary, shaping the product into the desired formulation.

Oral formulations wherein the carrier is a solid are most preferably presented as unit dose formulations such as boluses, capsules or tablets each containing a predetermined amount of active compound. A tablet may be made by compression or moulding, optionally with one or more accessory ingredients. Compressed tablets may be prepared by compressing in a suitable machine an active compound in a free-flowing form such as a powder or granules optionally mixed with a binder, lubricant, inert diluent, lubricating agent, surface-active agent or dispersing agent. Moulded tablets may be made by moulding an active compound with an inert liquid diluent. Tablets may be optionally coated and, if uncoated, may optionally be scored. Capsules may be prepared by filling an active compound, either alone or in admixture with one or more accessory ingredients, into the capsule shells and then sealing them in the usual manner. Cachets are analogous to capsules wherein an active compound together with any accessory ingredient(s) is sealed in a rice paper envelope. An active compound may also be formulated as dispersible granules, which may for example be suspended in water before administration, or sprinkled on food. The granules may be packaged, e.g., in a sachet.

Formulations suitable for oral administration wherein the carrier is a liquid may be presented as a solution or a suspension in an aqueous or non-aqueous liquid, or as an oil-in-water liquid emulsion.

Oral formulations may include controlled release dosage forms, e.g., tablets wherein an active compound is formulated in an appropriate release-controlling matrix, or is coated with a suitable release-controlling film. Such formulations may be particularly convenient for prophylactic use.

Pharmaceutical formulations suitable for rectal administration wherein the carrier is a solid are most preferably presented as unit dose suppositories. Suitable carriers include cocoa butter and other materials commonly used in the art. The suppositories may be conveniently formed by admixture of an active compound with the softened or melted carrier(s) followed by chilling and shaping in moulds.

Pharmaceutical formulations suitable for parenteral administration include sterile solutions or suspensions of an active compound in aqueous or oleaginous vehicles.

Injectable preparations may be adapted for bolus injection or continuous infusion. Such preparations are conveniently presented in unit dose or multi-dose containers, which are sealed after introduction of the formulation until required for use. Alternatively, an active compound may be in powder form, which is constituted with a suitable vehicle, such as sterile, pyrogen-free water, before use.

An active compound may also be formulated as long-acting depot preparations, which may be administered by intramuscular injection or by implantation, e.g., subcutaneously or intramuscularly. Depot preparations may include, for example, suitable polymeric or hydrophobic materials, or ion-exchange resins. Such long-acting formulations are particularly convenient for prophylactic use.

Formulations suitable for pulmonary administration via the buccal cavity are presented such that particles containing an active compound and desirably having a diameter in the range of 0.5 to 7 microns are delivered in the bronchial tree of the recipient.

As one possibility such formulations are in the form of finely comminuted powders which may conveniently be presented either in a pierceable capsule, suitably of, for example, gelatin, for use in an inhalation device, or alternatively as a self-propelling formulation comprising an active compound, a suitable liquid or gaseous propellant and optionally other ingredients such as a surfactant and/or a solid diluent. Suitable liquid propellants include propane and the chlorofluorocarbons, and suitable gaseous propellants include carbon dioxide. Self-propelling formulations may also be employed wherein an active compound is dispensed in the form of droplets of solution or suspension.

Such self-propelling formulations are analogous to those known in the art and may be prepared by established procedures. Suitably they are presented in a container provided with either a manually-operable or automatically functioning valve having the desired spray characteristics; advantageously the valve is of a metered type delivering a fixed volume, for example, 25 to 100 microlitres, upon each operation thereof.

As a further possibility, an active compound may be in the form of a solution or suspension for use in an atomizer or nebuliser whereby an accelerated airstream or ultrasonic agitation is employed to produce a fine droplet mist for inhalation.

Formulations suitable for nasal administration include preparations generally similar to those described above for pulmonary administration. When dispensed such formulations should desirably have a particle diameter in the range 10 to 200 microns to enable retention in the nasal cavity; this may be achieved by, as appropriate, use of a powder of a suitable particle size or choice of an appropriate valve. Other suitable formulations include coarse powders having a particle diameter in the range 20 to 500 microns, for administration by rapid inhalation through the nasal passage from a container held close up to the nose, and nasal drops comprising 0.2 to 5% w/v of an active compound in aqueous or oily solution or suspension.

It should be understood that in addition to the aforementioned carrier ingredients the pharmaceutical formulations described above may include, an appropriate one or more additional carrier ingredients such as diluents, buffers, flavouring agents, binders, surface active agents, thickeners, lubricants, preservatives (including anti-oxidants) and the like, and substances included for the purpose of rendering the formulation isotonic with the blood of the intended recipient.

Pharmaceutically acceptable carriers are well known to those skilled in the art and include, but are not limited to, 0.1 M and preferably 0.05 M phosphate buffer or 0.8% saline. Additionally, pharmaceutically acceptable carriers may be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Preservatives and other additives may also be present, such as, for example, antimicrobials, antioxidants, chelating agents, inert gases and the like.

Formulations suitable for topical formulation may be provided for example as gels, creams or ointments. Such preparations may be applied e.g. to a wound or ulcer either directly spread upon the surface of the wound or ulcer or carried on a suitable support such as a bandage, gauze, mesh or the like which may be applied to and over the area to be treated.

Liquid or powder formulations may also be provided which can be sprayed or sprinkled directly onto the site to be treated, e.g. a wound or ulcer. Alternatively, a carrier such as a bandage, gauze, mesh or the like can be sprayed or sprinkle with the formulation and then applied to the site to be treated.

In some embodiments, pharmaceutical formulations of the invention are particularly suited for ophthalmic administration, which is directly administered to the eye.

In some embodiments, such ophthalmic formulations may be administered topically with eye drops. In other embodiments, the ophthalmic formulations may be administered as an irrigating solution. In other embodiments, the ophthalmic formulations may be administered periocularly. In other embodiments, the ophthalmic formulations may be administered intraocularly.

In another teaching, the disclosure provides a topical, periocular, or intraocular ophthalmic formulation useful for neuroprotection and/or neuroregeneration in a subject suffering from or at risk of ocular impairment or vision loss due to neural damage.

Topical ophthalmic formulations administered in accordance with the present disclosure may also include various other ingredients including, but not limited to, surfactants, tonicity agents, buffers, preservatives, cosolvents, and thickeners.

A topical ophthalmic formulation administered topically, periocularly or intraocularly comprises an ophthalmically effective amount of one or more Chk2 inhibitors as described herein. As used herein, an “ophthalmically effective amount” is an amount sufficient to reduce or eliminate the signs or symptoms of an ocular condition described herein. In general, for formulations intended for topical administration to the eye in the form of eye drops or eye ointments, the total amount of active agent may be 0.001 to 1.0% (w/w). When applied as eye drops, 1-2 drops (approximately 20-45 μl each) of such formulations may be administered once to several times a day.

One route of administration is local. The compounds of the present disclosure can be administered as solutions, suspensions, or emulsions (dispersants) in an ophthalmically acceptable vehicle. An “ophthalmically acceptable” component, as used herein, refers to a component that does not cause any significant eye damage or discomfort over the intended concentration and intended use time. Solubilizers and stabilizers should be non-reactive. “Ophthalmically acceptable vehicle” refers to any substance or combination of substances that is non-reactive with the compound and suitable for administration to a patient. Suitable vehicles include physiologically acceptable oils such as silicone oil, USP mineral oil, white oil, poly (ethylene-glycol), polyethoxylated castor oil and vegetable oils such as corn oil or peanut oil Can be a non-aqueous liquid medium. Other suitable vehicles may be aqueous or oil-in-water solutions suitable for topical application to the patient's eye. These vehicles can preferably be based on ease of formulation and the ease with which a patient can administer such formulations due to the instillation of 1-2 drops of solution onto the affected eye. Formulations can also be suspensions, viscous or semi-viscous gels, or other types of solid or semi-solid formulations, and fatty bases (natural waxes such as beeswax, carnauba wax, wool wax (wool oil) (Wool fat)), refined lanolin, anhydrous lanolin); petroleum wax (eg, solid paraffin, microcrystalline wax); hydrocarbon (eg, liquid paraffin, white petrolatum, yellow petrolatum); or combinations thereof). The formulation can be applied manually or by use of an applicator (such as a wipe, contact lens, dropper, or spray).

Various tonicity agents can be used to adjust the tonicity of the composition, preferably to that of natural tears for ophthalmic compositions. For example, sodium chloride, potassium chloride, magnesium chloride, calcium chloride, dextrose, and/or mannitol can be added to the composition to approximate physiological tonicity. The amount of such isotonic agent will vary depending on the particular agent to be added. In general, however, the formulation will have a sufficient amount of tonicity agent so that the final composition has an osmolality that is ophthalmically acceptable (generally about 200-400 mOsm/kg).

Other agents may also be added to the topical ophthalmic formulation of the present disclosure to increase the viscosity of the carrier. Examples of viscosity enhancing agents include, but are not limited to: polysaccharides (such as hyaluronic acid and its salts, chondroitin sulfate and its salts, dextran, polymers of various cellulose families); vinyl polymers; and acrylics Acid polymer. In general, a phospholipid carrier or artificial tear carrier composition exhibits a viscosity of 1 to 400 centipoise.

An appropriate buffer system (eg, sodium phosphate, sodium acetate, sodium citrate, sodium borate, or boric acid) can be added to the formulation to prevent pH fluctuations under storage conditions. The specific concentration will vary depending on the agent used. However, preferably the buffer is selected to maintain a target pH within the range of pH 6 to 7.5.

Formulations of the disclosure may be administered intraocularly after a traumatic event involving retinal tissue and optic nerve head tissue or before or during ophthalmic surgery to prevent injury or damage. Formulations useful for intraocular administration are generally intraocular injection formulations or surgical washes.

Compounds and formulation of the present disclosure may also be administered by periocular or intraocular administration and can be formulated in a solution or suspension for periocular/intraocular administration. The compounds/formulations of the disclosure may be administered periocularly/intraocularly after traumatic events involving retinal tissue and optic nerve head tissue or before or during ophthalmic surgery to prevent injury or damage. Formulations useful for periocular/intraocular administration are generally in the form of injection formulations or surgical lavage fluids.

Periocular administration refers to administration to tissues near the eye (such as administration to tissues Or spaces around the eyeball and in the orbit). Periocular administration can be performed by injection, deposition, or any other mode of placement. Periocular routes of administration include, but are not limited to, subconjunctival, suprachoroidal, near sclera, near sclera, subtenon, subtenon posterior, retrobulbar, periocular, or extraocular delivery. Intraocular delivery refers to administration directly into the eye, such as by way of injection, or by way of a depot surgically inserted into the eye, for example.

Therapeutic formulations for veterinary use may be in any of the above-mentioned forms, but conveniently may be in either powder or liquid concentrate form. In accordance with standard veterinary formulation practice, conventional water-soluble excipients, such as lactose or sucrose, may be incorporated in the powders to improve their physical properties. Thus, particularly suitable powders of this invention comprise 50 to 100% w/w and preferably 60 to 80% w/w of the active ingredient(s) and 0 to 50% w/w and preferably 20 to 40% w/w of conventional veterinary excipients. These powders may either be added to animal feedstuffs, for example by way of an intermediate premix, or diluted in animal drinking water.

Liquid concentrates of this invention suitably contain the compound or a derivative or salt thereof and may optionally include a veterinary acceptable water-miscible solvent, for example, polyethylene glycol, propylene glycol, glycerol, glycerol formal or such a solvent mixed with up to 30% v/v of ethanol. The liquid concentrates may be administered to the drinking water of animals.

In one embodiment according to this disclosure, the pharmaceutical formulation is presented in a form suitable for oral administration.

The present disclosure will now be described by way of example.

### Materials and Methods

### Experimental Design

The aim of this study was to determine the role of ATM inhibition using a brain penetrant ATM inhibitor, AZD1390, on recovery after spinal cord injury. Cell culture experiments using primary adult rat DRGN were subjected to ATM inhibitors including AZD1390 and KU-60019 and DRGN survival and neurite outgrowth was assessed in the presence of inhibitor concentration of CNS myelin extracts to mimic the post-injury environment of the spinal cord. All experiments were performed with the investigator masked to the treatment conditions and unmasked after analysis. We then used our well-characterized in vivo DC injury model of SCI in mice to determine the effect of ATM inhibition on axon regeneration and functional recovery. In vivo sample sizes were determined at the outset and based on power calculations derived from previous similar experiments in our laboratories. Animals were then randomly assigned to treatment groups roughly containing equal numbers of male and female mice and experimenters were masked to the treatment and procedural conditions. No animals were excluded for any reason no expected or unexpected adverse events were encountered. All animals' tissue samples were processed at the same time and analysed to prevent batch effects. Animals were housed in groups of four animals/cage in the same facility and the number of biological replicates is indicated in the figure legends.

### Animals

All animal experiments were licensed by the UK Home Office and approved by the University of Birmingham's Animal Welfare and Ethical Review Board. Surgical procedures were carried out accordance to the guidelines of the UK Animals Scientific Procedures Act, 1986, the Revised European Directive 1010/63/EU and conformed to the guidelines and recommendation of the use of animals by the Federation of the European Laboratory Animal Science Associations (FELASA). Experiments also conformed to the Animal Research: Reporting of in vivo Experiments (ARRIVE) guidelines. Adult male and female 6-8-week-old Sprague-Dawley rats weighing 170-220 g (Charles River, Margate, UK) were used in all experiments. Animals were housed in a standard animal facility maintained at 21° C. and operating a 12-hour light-dark cycle, with free access to food and water. After surgery, animals were returned to their home cages and pre- and post-operative analgesia was provided as standard and as recommended by the named veterinary surgeon.

### Primary Adult Rat DRGN Cultures

Primary adult rat DRGN cultures were prepared as described by us previously (11). DRGN were dissociated using collagenase and cultured in supplemented Neurobasal-A (#10888022; NBA) containing B27 supplement (#17504044), L-glutamine (#25030081) and gentamicin (#15710064) (all from Invitrogen, Paisley, UK) at a plating density of 500/well in 8-well chamber slides (#C6932; Beckton Dickinson, Oxford, UK) pre-coated with 100 μg/ml poly-D-lysine (#P6407; Sigma, Poole, UK). Glial cell proliferation was inhibited in cultures using 5-fluoro-2-deoxyuridine (5-FDU; #343333; Sigma) at 30 μM (11). To mimic the inhibitory environment of the CNS, myelin extracts (CME; CNS myelin extracts) were prepared from adult Sprague-Dawley rats as described by us previously (11), confirmed to contain significant MBP, Nogo-A, MAG and Brevican and used at 200 μg/ml to completely inhibit DRGN neurite outgrowth (11). Positive controls included pre-optimized fibroblast growth factor-2 (FGF-2) (#100-18C, Peprotech, London, UK; 10 ng/ml (11)). Cells were cultured for 4 days in a humidified chamber at 37° C. and 5% CO₂ before being subjected to immunocytochemistry, as described below. Treatments were added in triplicate and repeated on 3 independent occasions (i.e. total n=9 wells/treatment) by an investigator masked to the treatment conditions.

### ATM Inhibitors

AZD1390 (a gift from AstraZeneca) and KU-60019 (#4176; Tocris, Oxford, UK) were dissolved in DMSO (Sigma, Poole, UK) at stock concentrations of 10 mM for in vitro use. Stock concentrations were then diluted to the appropriate concentrations for in vitro use in supplemented NBA or in vehicle solution comprising 0.5% (w/v) hydroxypropyl methylcellulose (HPMC) and 0.1% (w/v) Tween 80 for in vivo oral use (Durant, 2018]. For intrathecal delivery of KU-60019, stocks were diluted to the final doses in phosphate buffered saline (7).

### Immunocytochemistry and Quantification of DRGN Survival and Neurite Outgrowth

DRGN were fixed in 8-well chamber slides in situ using 4% paraformaldehyde before being subjected to immunocytochemistry with rabbit anti-βIII tubulin antibodies (#T3952; 1:200 dilution; Sigma) to detect DRGN soma and neurites, as described previously (11). Alexa-488 goat anti-mouse IgG (#A32723; 1:400 dilution; ThermoFisher, Leicester, UK) secondary antibodies were used to visualize DRGN soma and neurites using an Axioplan 2 epifluorescent microscope equipped with an AxioCam HRc and running Axiovision Software (all from Carl Zeiss, Hertfordshire, UK).

With the investigator masked to the treatment conditions, the proportion of βIII-tubulin⁺ DRGN, the number of DRGN with neurites and the longest DRGN neurite length were all calculated using Axiovision Software, as described by us previously (11).

### Dorsal Column Crush Injury Model of SCI

Experiments comprised n=6 mice/group: (1), Sham uninjured control group (anaesthesia followed by partial laminectomy but no DC crush injury); (2), DC crush injury+oral vehicle solution (0.5% HPMC and 0.1% Tween 80); (3), DC crush injury+oral AZD1390; and (4), DC crush injury+oral KU-60019. A final group of animals received intrathecal injection of KU-60019 every 24 hours through a cannula implanted into the subarachnoid space (7).

Mice were subcutaneously injected with Buprenorphine and anaesthetized using 5% isoflurane and 1.8 l/min O₂. After a partial laminectomy at T8, DC were crushed bilaterally using calibrated watchmaker's forceps as described by us previously (12). Animals were dosed immediately after injury and allowed to recover in their home cage. Drugs were given every 24 hours for the duration of each experiment.

For western blot, animals were killed at 4 weeks and tissues harvested as described below. For immunohistochemistry and behavioural analysis, animals were treated up to 6 weeks after DC injury.

### Cholera Toxin B Labelling and Quantification of DC Regenerating Axons

To retrogradely trace regenerating DC axons, 1% Cholera toxin B (CTB; #104, List Biologicals, Campbell, CA, USA) was injected into the sciatic nerve at mid-thigh levels, using glass microneedles, 1 week before killing mice with rising concentrations of CO₂. Animals were intracardially perfused with 4% paraformaldehyde and CTB labelled axons were detected by immunohistochemistry using a goat polyclonal anti-CTB antibody (#703; 1:1000 dilution, List Biological Labs) at 6 weeks after injury, as described later.

CTB⁺ regenerating axons were quantified in sagittal sections of the spinal cord from the whole series of tissues for each mouse (total n=12 mice/group). CTB intensity was quantified in ImageJ software (www.imagej.nih.gov) at different distance rostral to the injury center and expressed as a % of CTB intensity caudal to the injury site, which controls for slight variations in tracing efficiency.

### Tissue Harvesting

Methods for tissue harvesting have been described by us previously (7, 12). Briefly, for western blot analysis: animals were killed by rising concentrations of CO₂ and L4/L5 DRG pairs were dissected out and snap frozen in liquid nitrogen and stored at −80° C. until required (7). For immunohistochemistry: animals were killed in rising concentrations of CO₂ and intracardially perfused with 4% paraformaldehyde (7, 12). The SCI site+5 mm either side of the lesion center was dissected out and postfixed in 4% formaldehyde for 2 hr, followed by cryoprotection in a graded series of sucrose and embedded in optimal temperature cutting medium (#12678646, ThermoFisher). Sections were cut at 15 μm-thick stored at −80° C. until required.

### Western Blot Analysis and Densitometry

Total protein was extracted from DRGN cultures and fresh DRG tissues as described by us previously (7, 12). Briefly, cultures/DRG tissues were washed in PBS and total protein extracted in ice-cold lysis buffer containing 20 mM HEPES, 1 mM EDTA, 150 mM NaCl, 1% NP-40 and 1 mM DTT and supplemented with protease (#P8340) and phosphatase inhibitor cocktails (#P5726) (all from Sigma). After a protein assay using the Bradford Assay Kit (#5000001; BioRad, Watford, UK), 15 μg of total protein was separated on 12% Tris-glycine SDS-PAGE gels. Proteins were then transferred onto PVDF membranes (#1EVH00005, Merck, Gillingham, UK), blocked in non-fat milk and incubated with rabbit anti-pATM (ser1981) antibody (#13050, 1:200 dilution, Cell Signalling Technology, London, UK) overnight at 4° C. Membranes were then washed in Tris buffered saline (TBS) and incubated with appropriate mouse/rabbit HRP-labelled secondary antibody (#GENA934; 1:1000 dilution, GE Healthcare, Buckinghamshire, UK) and bands detected using an enhanced chemiluminescence kit (#RPN2108, GE Healthcare). Rabbit anti-β-actin antibody (#ZRB1312, 1:1000; Sigma) was used as a loading control for western blots.

For densitometry, western blots were scanned into Adobe Photoshop (Adobe Systems, San Jose, CA, USA) keeping all scanning parameters the same between each blot and densitometrically analysed using the built-in gel plotting macros in ImageJ (ww.nihimage.nih.gov), as previously described (7).

### Immunohistochemistry

Sections were thawed at room temperature and after washes in PBS, permeabilized in PBS containing 0.1% Triton X-100, blocked in PBS containing 3% bovine serum albumin and 0.05% Tween-20 (all from Sigma) and incubated in primary antibodies, as described by us previously (7).

### Electrophysiology

Compound action potentials (CAP) were recorded at 6 weeks after DC injury and treatment as described by us previously (7). Briefly, with the experimenter masked to the treatment conditions, CAPs across the surface of the lesion site were recorded using silver wire electrodes stimulated at lumbar (L1)-L2 and CAPs recorded at cervical (C)4-C5. Spike 2 software (Cambridge Electronic Design, Cambridge, UK) was used to process and analyse the traces and determine the CAP amplitude and CAP area for each condition.

Functional Tests after DC Injury and Treatment

Tape sensing and removal and horizontal ladder crossing tests were used to detect sensory and locomotor changes after DC injury and treatment as described by us previously (7). Briefly, tape sensing and removal was tested by attaching a 15/15 mm piece of sticky tape (Kip Hochkrepp, Bocholt, Germany) onto the plantar surface of each the left paw of each mouse and recording the time taken to detect and remove the tape. For the ladder crossing test, each mouse was trained prior to DC injury to master the traversing the ladder (0.9 m long and 15.5 cm wide with randomly adjusted rungs). Animals were assessed crossing the ladder and the total number of steps and the number of left and right hind paw slips were recorded and presented as an error ratio.

All of these tests were performed at 2 days after injury, then at 1 week followed by weekly tests for 6 weeks. The experimenter was masked to the treatment conditions and animals were assessed in the same order and time of day with each test performed for 3 individual trials on each occasion.

### Statistical Analysis

All results are presented as mean±standard error of the mean (SEM). Statistical significance was calculated by one-way analysis of variance (ANOVA) with post-hoc Dunnett's method using SPSS Statistics 19 (IBM, New York, USA). For the horizontal ladder crossing and tape removal tests, data was analysed as described previously (7) using R statistics package (www.r-project.org). Briefly, whole time-course of lesioned and sham-treated animals in the ladder crossing test was compared using binomial generalized linear mixed models (GLMM), fitted in R using Ime4 plugin with the gImer plugin function and P values calculated using parametric bootstrap. For the tape sensing and removal test, linear mixed models (LMM) were calculated by model comparison in R using the pbkrtest plugin, with the Kenward-Roger method.

### Drosophila Methods

Male Drosophila melanogaster carrying a UAS-(GGGGCC)₁₀₈ transgene were crossed to virgin females of the w^{DAH};Elav-GS steroid-inducible Gal4 driver line. After eclosion as adults, flies of the correct genotype were transferred to food containing 200 μM mifepristone to induce expression of GGGGCC specifically in adult neurons. UAS-shRNA (TRiP.GL00138) was co-expressed under the control of the same driver to knockdown expression of Drosophila tefu (the ATM homologue). Virgin females of the driver line were crossed to w¹¹¹⁸ males or TRiP.GL00138 males as controls.

### Tracking of Movement

The startle response of males flies was quantified as described in Taylor and Tuxworth, 2019 except 1. the flies were reared at a constant 25° C. and 2. The food contained 200 μM mifepristone to maintain expression from the UAS-GGGGCC transgene. The rates of decline in performance index were compared by ANOVA with a Tukey's multiple comparison test. A significance threshold was set to p<0.05.

### Survival Analysis

45-60 adult flies per genotype were housed in cohorts of 10-20 on food containing 200 μM mifepristone. Flies were provided fresh food and deaths scored 2-3 times per week. Comparisons of median survival was by Log Rank test.

### Results

### AZD1390 Suppresses ATM and Promotes DRGN Survival and Neurite Outgrowth In Vitro

Adult rat DRGN cultures have been employed by us as a useful screen for neuroprotective and neurite outgrowth promoting factors (7, 11). CNS myelin extracts (CME) containing Nogo-A, myelin associated glycoprotein and chondroitin sulphate proteoglycans, may also be carefully titred and added to these cultures to mimic a CNS injury environment and, therefore, the disinhibition of neurite outgrowth can be assessed (11).

Treatment of DRGN cultures with increasing concentrations of AZD1390 from 1-10 nM, significantly reduced the levels of ATM activation (phosphorylated ATM (pATM) levels), with 5 nM being the lowest most effective dose which reduced pATM levels by 70% compared to vehicle-treated (0 nM AZD1390) cultures (FIGS. 1A and B). Increasing the concentrations of AZD1390 beyond 5 nM did not cause further suppression of pATM levels in DRGN cultures (FIGS. 1A and B). Suppression of pATM with 5 nM AZD1390 increased DRGN survival by 60%, increased the percentage of DRGN with neurites by 88% and increased the longest neurite length by 81% when each was compared to vehicle-treated DRGN controls (FIG. 1C-E). The positive control, fibroblast growth factor-2 (FGF-2), was also inferior compared to AZD1390 in promoting DRGN survival and neurite outgrowth. In addition, we used KU-60019, another tool ATM inhibitor, at the same concentrations as used in a recent study (7) and showed similar levels of DRGN survival, % DRGN with neurites and the mean longest neurite length as observed with AZD1390 (FIG. 2C-F). These results suggest that inhibition of pATM in vitro using either AZD1390 or KU-60019 promotes similar levels of significant DRGN survival and disinhibited neurite outgrowth.

Brain Penetrant AZD1390 Promotes DC Axon Regeneration after SCI

We next employed a moderate severity mouse dorsal column (DC) injury model of SCI, which affects long-tract ascending fibers, to investigate if AZD1390 can promote axon regeneration in vivo (7, 12). Since an effective dose of 20 mg/kg was already reported for optimal brain exposure of AZD1390 (9), we first attempted to optimize oral delivery of KU-60019. Despite the use of relatively high oral doses of KU-60019 up to 100 mg/kg, we observed no pATM inhibition in DRGN from SCI treated animals (Supplementary FIG. 1). However, in separate experiments intrathecal delivery of KU-60019 after injury suppressed pATM levels by 81% (Supplementary FIG. 1), consistent with our previously published report showing significant suppression of DNA damage foci using γH2Ax staining after intrathecal delivery of KU-60019 in the same SCI model (7). These results demonstrate that KU-60019 was not available at high enough concentrations after oral delivery to engage the target in the spinal cord and cause pATM suppression.

Using a previously characterised oral dose of 20 mg/kg AZD1390 delivered once daily (9) for the duration of our experiment. We observed significant suppression of pATM (pSer 1981) levels, whilst the same dose of oral KU-60019 had no effect on pATM levels (FIGS. 2A and B). Consistent with the suppression of pATM levels by AZD1390, Cholera Toxin B tracing of axons showed that only oral AZD1390 but not oral KU-60019 promoted DC axon regeneration through the lesion site and into the rostral cord with up to 12% of axons regenerating up to 1 mm beyond the lesion site compared to vehicle-treated mice (FIGS. 2C and D). These results demonstrate that only oral AZD1390 promotes DC axon regeneration.

To determine if the axon regeneration we observed after oral AZD1390 is useful in the return of function after SCI, we first used electrophysiology to record compound action potentials (CAPs) across the lesion site (7). Spike 2 software processed CAP traces showed that after DC injury, the normal CAP trace was ablated (FIG. 3A). However, only oral AZD1390 treatment and not KU-60019, showed return of a significant CAP trace (FIG. 3A). All of the normal CAP traces were abolished at the end of the recording phase by dorsal hemisection to demonstrate technical success of this technique (FIG. 3A). CAP amplitudes (FIG. 3B) and CAP areas (FIG. 3C) were also significantly improved by oral AZD1390 and not KU-60019.

We then determined if inhibition of pATM using oral AZD1390 promoted sensory and locomotor function after SCI. We used a tape sensing and removal test to determine sensory function. Animal treated with oral AZD1390 removed the tape in significantly shorter time than controls but again KU-60019 had no effect (FIG. 3D). Indeed, sensory function in animals treated with oral AZD1390 returned to the baseline of sham-uninjured animals within 4 weeks after injury. In contrast, a significant deficit remained for the full 6 weeks of the experiment in animals treated with oral KU-60019- or in DC+vehicle-treated animals, (FIG. 3D). Locomotor function, as assessed by a ladder crossing test, also showed marked improvement in AZD1390-treated animals, compared to KU-60019- or vehicle-treated animals (FIG. 3E). AZD1390-treated animals recovered to sham-uninjured control levels by 4 weeks, whilst a significant deficit remained in KU-60019- or vehicle-treated animals (FIG. 3E).

Take together the results demonstrate that oral AZD1390 but not KU-60019 promotes significant electrophysiological, sensory and locomotor function recovery after SCI and that this recovery is consistent with suppression of pATM.

In further experiments, Quantification of movement and lifespan in a Drosophila melanogaster model of ALS-FTD was studied. Drosophila were engineered to express repeats of GGGGCC (G₄C₂) specifically in neurons of adult flies. GGGGCC repeats model the intronic expansion in the C9orf72 locus that is the most common genetic form of ALS-FTD. The results in FIG. 5, show that targeting ATM expression is protective in an in vivo model of ALS-FTD. Thus, it is expected that ATM inhibitor compounds, such as the compounds claimed herein, will be useful in the treatment of conditions associated with ATM activity, such as ALS and ALS-FTD.

### Discussion

In this study, we have demonstrated that oral delivery of a potent, highly selective and CNS-penetrant ATM inhibitor, AZD1390, is able to stimulate significant functional recovery after spinal cord injury. AZD1390 was able to promote axon regeneration, electrophysiological improvements across the lesion site and improved sensory and locomotor function after DC injury in vivo, all consistent with suppression of ATM activation (i.e. pATM). Although KU-60019 promoted similar levels of DRGN survival and disinhibited neurite outgrowth as AZD1390 in our in vitro survival and neurite regeneration assay, it was unable to reduce pATM levels in vivo even when delivered orally at high doses. Consistent with the in vitro results, KU-60019 delivered orally had no effect on functional recovery after DC injury in vivo. In contrast, intrathecal delivery of KU-60019 after SCI in vivo engaged the target and was able to suppress pATM levels significantly.

AZD1390 possesses favorable physical, chemical and PK/PD properties for clinical applications requiring exposures in the CNS (9). It inhibits ATM activity with an IC₅₀ of 0.5-3 nM and can modulate the DNA damage response.

We show that AZD1390 engages with the target at nM concentrations in vitro and enhanced adult DRGN survival and disinhibited DRGN neurite outgrowth, increasing the proportion of DRGN with neurites and the longest DRGN neurite length. This suggests that inhibition of ATM activation promotes survival and neurite growth initiation and elongation, all believed to be regulated by different pathways in the CNS. For example, suppression of caspase-2 promoted >95% retinal ganglion cell survival (RGC) but did not promote axon regeneration unless combined with a neurite growth promoting stimulus (13). Without wishing to be bound by theory, we speculate that both axon regenerative and survival signaling pathways are modulated by ATM inhibition.

AZD1390 was also able to promote significant DC axon regeneration after SCI in vivo, together with improvements in CAP traces across the lesion site and significant sensory and locomotor function. Sensory and locomotor function were so remarkable that after 4 weeks of treatment, animals were indistinguishable from sham-treated control animals, whilst a significant deficit remained in the vehicle- and ATR inhibitor-treated animals. This demonstrates that AZD1390 could potentially be a novel treatment after SCI that restores lost sensory and locomotor function.

KU-60019, a tool inhibitor of ATM with an IC₅₀ of 6.3 nM was also able to suppress ATM activation in vitro and promote significant DRGN survival and neurite outgrowth. However, the fact that KU-60019 was unable to suppress pATM levels in DRGN after oral delivery suggests that KU-60019 is unable to access the CNS in high enough concentrations, despite the BSCB being compromised for at least 21 days after SCI (14). Intriguingly, KU-60019 was able to suppress ATM activation in DRGN after intrathecal delivery. This is consistent with our previous study where we showed that the same concentration of KU-60019 as used here and delivered via intrathecal injection was able to suppress γH2Ax levels, a marker of DNA damage, and promote axon regeneration and functional recovery in the same SCI model (7). The current study shows that KU-60019 is only available in sufficient quantities to suppress ATM activation after intrathecal delivery.

Intrathecal delivery represents a useful delivery route for some drugs and has obvious advantages as it gives direct access to the CNS, often requiring lower drug doses and thus limiting unwanted side-effects (15). However, at present only a few drugs are delivered intrathecally in the clinic and mainly for analgesic purposes (15). Therefore, safe delivery via intrathecal delivery requires further optimization. Since AZD1390 can be delivered orally and reaches the brain in sufficient quantities to inhibit ATM activation, it represents a significant advantage in terms of delivery to the spinal cord or brain.

Stimulation of recovery may require only a brief period of treatment, and potential side-effects would be minimized. In addition, it is expected that 75% inhibition of pATM may not be necessary, since 50% reduction in the levels of γH2Ax was sufficient to promote similar levels of axon regeneration and functional recovery (7).

In conclusion, our study shows that nM concentrations of AZD1390 delivered orally and hence must cross the BBB, promote significant restoration of function after SCI. Moreover, evidence that targeting ATM expression is protective in other neurological conditions, such as ALS, supports the view that the compounds as described herein, will have broad application in treating conditions which are associated with ATM activity.

### REFERENCES

1. Y. D. Kang, H.; Zhou, H.; Wei, Z.; Liu, L.; Pan, D.; Feng, S., Epidemiology of worldwide spinal cord injury: a literature review. Journal of Neurorestoratology 2018, 61-69 (2018).
2. J. W. Fawcett, The Struggle to Make CNS Axons Regenerate: Why Has It Been so Difficult? Neurochem Res 45, 144-158 (2020).
3. A. Uyeda, R. Muramatsu, Molecular Mechanisms of Central Nervous System Axonal Regeneration and Remyelination: A Review. Int J Mol Sci 21, (2020).
4. K. Aziz et al., Targeting DNA damage and repair: embracing the pharmacological era for successful cancer therapy. Pharmacol Ther 133, 334-350 (2012).
5. B. J. Lamarche, N. I. Orazio, M. D. Weitzman, The MRN complex in double-strand break repair and telomere maintenance. FEBS Lett 584, 3682-3695 (2010).
6. Y. Shiloh, Y. Ziv, The ATM protein kinase: regulating the cellular response to genotoxic stress, and more. Nat Rev Mol Cell Biol 14, 197-210 (2013).
7. R. I. T. Tuxworth, M. J.; Anduaga, A-M.; Hussien-Ali, A.; Chatzimatthaiou, S.; Longland, J.; Thompson, A. M.; Almutiri, S.; Alifragis, P.; Kyriacou, C. P.; Kysela, B.; Ahmed, Z., Attenuating the DNA damage response to double-strand breaks restores function in models of CNS neurodegeneration. Brain Communications 1, fcz005 (2019).
8. J. E. Simpson et al., A neuronal DNA damage response is detected at the earliest stages of Alzheimer's neuropathology and correlates with cognitive impairment in the Medical Research Council's Cognitive Function and Ageing Study ageing brain cohort. Neuropathol Appl Neurobiol 41, 483-496 (2015).
9. S. T. Durant et al., The brain-penetrant clinical ATM inhibitor AZD1390 radiosensitizes and improves survival of preclinical brain tumor models. Sci Adv 4, eaat1719 (2018).
10. Pike K G, et al. The identification of potent, selective, and orally available inhibitors of ataxia telangiectasia mutated (ATM) kinase: the discovery of AZD0156 (8-{6-[3-(dimethylamino)propoxy]pyridin-3-yl}-3-methyl-1-(tetrahydro-2H-pyran-4-yl)-1,3-dihydro-2H-imidazo[4,5-c]quinolin-2-one). J Med Chem 2018; 61:3823-41
11. Z Ahmed et al., Disinhibition of neurotrophin-induced dorsal root ganglion cell neurite outgrowth on CNS myelin by siRNA-mediated knockdown of NgR, p75NTR and Rho-A. Mol Cell Neurosci 28, 509-523 (2005).
12. S. Surey, M. Berry, A. Logan, R. Bicknell, Z. Ahmed, Differential cavitation, angiogenesis and wound-healing responses in injured mouse and rat spinal cords. Neuroscience 275, 62-80 (2014).
13. V. Vigneswara et al., Combined suppression of CASP2 and CASP6 protects retinal ganglion cells from apoptosis and promotes axon regeneration through CNTF-mediated JAK/STAT signalling. Brain 137, 1656-1675 (2014).
14. W. D. Whetstone, J. Y. Hsu, M. Eisenberg, Z. Werb, L. J. Noble-Haeusslein, Blood-spinal cord barrier after spinal cord injury: relation to revascularization and wound healing. J Neurosci Res 74, 227-239 (2003).
15. M. M. Bottros, P. J. Christo, Current perspectives on intrathecal drug delivery. J Pain Res 7, 615-626 (2014).
16. Matthew J. Taylor & Richard I. Tuxworth (2019) Continuous tracking of startled Drosophila as an alternative to the negative geotaxis climbing assay, Journal of Neurogenetics, 33:3, 190-198.

1. A method of treating a neurological condition by protecting against or treating neuronal damage or neuronal degeneration, the method comprising administering, to a subject in need thereof, a pharmaceutical formulation comprising a compound of Formula (I): 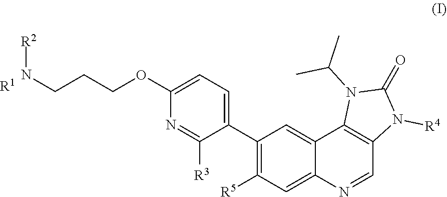
or a pharmaceutically acceptable salt, solvate, ester or other physiologically acceptable functional derivative thereof, where:
R¹ is methyl;
R² is hydro or methyl; or R¹ and R² together with the nitrogen atom to which they are bonded form an azetidinyl, pyrrolidinyl or piperidinyl ring;
R³ is hydro or fluoro;
R⁴ is hydro or methyl; and
R⁵ is hydro or fluoro; and
at least one pharmaceutically acceptable excipient,
wherein the pharmaceutical formulation is not applied directly to the central nervous system (CNS) and/or peripheral nervous system (PNS).

2. The method according to claim 1, wherein R¹ and R² together with the nitrogen atom to which they are bonded form a piperidinyl ring.

3. The method according to claim 1, wherein R³ is hydro.

4. The method according to claim 1, wherein R⁵ is fluoro.

5. The method according to claim 1, wherein R⁴ is methyl.

6. The method according to claim 1, wherein the compound is 8-[2-Fluoro-6-[3-(1-piperidyl)propoxy]-3-pyridyl]-1-isopropyl-3-methyl-imidazo[4,5-c]quinolin-2-one (AZD1390).

7. The method according to claim 1, wherein the method promotes neuronal regeneration.

8. The method according to claim 1, wherein the neurological condition affects the spinal cord, brain and/or optic nerve.

9. The method according to claim 1, wherein the neurological condition is due to neuronal damage, caused by physical means and/or by chemical means.

10. The method according to claim 9, wherein the physical means results from surgery or trauma.

11. The method according to claim 10, wherein the trauma to be treated is traumatic brain injury (TBI), chronic traumatic encephalopathy (CTE); traumatic injury to the spinal cord or eye caused by e.g. blunt force, puncture, compression, concussive damage or ballistic damage; Ischaemia affecting the central nervous system; and traumatic injury to the peripheral nervous system, such as traumatic injury affecting motor, sensory or autonomic nerves, and traumatic injury affecting peripheral glia.

12. The method according to claim 8, wherein the method prevents neuronal dysfunction and maintains neuronal function.

13. The method according to claim 12, wherein the method is in the treatment of Chemotherapy-induced neuropathic pain and Chronic ophthalmic disorders, such as glaucoma, age-related macular degeneration and diabetic retinopathy.

14. The method according to claim 8, for treating retinal ischemia, acute retinopathy associated with trauma, postoperative complications following eye surgery, traumatic optic neuropathy (TON); and damage related to laser therapy (including photodynamic therapy (PDT)), damage related to surgical light-induced iatrogenic retinopathy, and damage related to corneal transplantation and stem cell transplantation of ocular cells.

15. The method according to claim 8, for treating recessive neurodegenerative disorders and PNS disorders, such as amyotrophic lateral sclerosis-frontal temporal dementia spectrum disorders (ALS-FTD), including forms associated with an expansion of the C9orf72 locus and spinal muscular atrophy; lysosomal storage disorders with a neurological association such as neuronal ceroid lipofuscinosis (NCL); and Neurodegenerative disorders affecting the peripheral nervous system, such as Charcot-Marie-tooth disease, acute motor axonal neuropathy, diabetic neuropathy and Guillain-Barre syndrome.

16. The method according to claim 1, wherein the formulation completely inhibits or partially inhibits ATM kinase activity.

17. The method according to claim 16, wherein ATM kinase is inhibited by at least 30%, 40%, 50%, 60%, 70% 80% or 90%.

18. The method according to claim 1, wherein the formulation is administered orally, parenterally (e.g. intravenously, intra-arterially, intramuscularly, or subcutaneously), topically, nasally, pulmonarily, sublingually, vaginally, or rectally.

19. The method according to claim 18, wherein the pharmaceutical formulation is administered orally.
